(19) United States
(12) Patent Application Publication
(10) Pub. No.: US 2009/0312622 A1
(43) Pub. Date: Dec. 17, 2009

(54) Device And Method For Determining A Value Of A Physiological Parameter Of A Body Fluid

(76) Inventor: Werner Regittnig, Graz (AT)

Correspondence Address: SMITH FROHWEIN TEMPEL GREENLEE BLAHA, LLC, Two Ravinia Drive, Suite 700, ATLANTA, GA, 30346, US

(21) Appl. No.: 12/443,081

(22) PCT Filed: Jul. 31, 2007
(86) PCT No.: PCT/EP2007/006784

§ 371 Date: Aug. 31, 2009

Related U.S. Application Data
(60) Provisional application No. 60/827,257, filed on Sep. 28, 2006.

(30) Foreign Application Priority Data
Sep. 28, 2006 (EP) 06020407.0

(51) Int. Cl. A61B 5/145

(52) U.S. Cl. 600/366

(57) ABSTRACT

A device for determining a value of a physiological parameter of a body fluid of a body under investigation, the device comprising a fluid reservoir for containing a perfusate fluid, a body interface insertable into the body under investigation for a fluid communication between the fluid reservoir and the body fluid, a fluid conduit between the fluid reservoir and the body interface, a bidirectional fluid transport unit for transporting the perfusate fluid from the fluid reservoir through the fluid conduit a the body interface into the body under investigation and for subsequently transporting a mixture of the perfusate fluid and the body fluid from the body under investigation through the body interface into the fluid conduit, and a sensor for sensing the value of the physiological parameter based on an analysis of the mixture of the perfusate fluid and the body fluid.

The invention relates to a device for determining a value of a physiological parameter of a body fluid of a body under investigation.

The invention further relates to a method for determining a value of a physiological parameter of a body fluid of a body under investigation.

In diabetes, the tight control of glucose metabolism is lost because the release of insulin from the beta cells of the pancreas is lacking—so-called “type 1 diabetes”—or abnormal—so-called “type 2 diabetes” (see Mathis, D, Vence, L and Benoist, C “β-Cell death during progression to diabetes”, Nature 414:792-798, 2001; Bell, G I, and Polonsky, K S “Diabetes mellitus and genetically programmed defects in beta-cell function”, Nature 414:788-791, 2001).

Thus, people with type 1 diabetes have to administer insulin from external sources for survival. People with type 2 diabetes are usually not dependent on exogenous insulin administration, but may require it for control of blood glucose levels if this is not achieved with diet alone or with oral hypoglycemic drugs (see Moller, D E “New drug targets for type 2 diabetes and the metabolic syndrome”, Nature 414:821-827, 2001). Administration of exogenous insulin by means of the subcutaneous route provides the basis of the current insulin therapy (see Owens, D R “New horizons—alternative routes for insulin therapy”, Nat Rev Drug Discov 1:529-540, 2002).

In the majority of the insulin-requiring diabetic patients, insulin is administered in the form of a bolus subcutaneous injection. However, an increasing number of patients is using external pumps to administer insulin in the form of a continuous subcutaneous infusion, so-called “insulin pump therapy” (see Owens, D R “New horizons—alternative routes for insulin therapy”, Nat Rev Drug Discov 1:529-540, 2002; Lenhard, M I, and Reeves, G D “Continuous subcutaneous insulin infusion: a comprehensive review of insulin pump therapy”, Arch Intern Med 161:2293-2300, 2001). In the insulin pump therapy, a transcutaneous indwelling catheter is inserted by the patient into the subcutaneous tissue of the abdomen, fixed with an adhesive strip, and connected to an insulin pump using a flexible tube. Typical transcutaneous indwelling catheter is composed of a soft cannula, which is introduced into the subcutaneous tissue with the aid of a metal needle and, after removal of the metal needle, may dwell over two to three days in the tissue.

With regard to glucose monitoring, large prospective clinical studies have suggested that tight blood glucose control is essential to minimize micro- and macrovascular complications in both type 1 and type 2 diabetes (see The Diabetes Control and Complications Trial Research Group “The effect of intensive treatment of diabetes on the development and progression of long-term complications in insulin-dependent diabetes mellitus”, N Engl J Med 329:977-986, 1993; UK Prospective Diabetes Study (UKPDS) Group “Intensive blood-glucose control with sulphonylureas or insulin compared with conventional treatment and risk of complications in patients with type 2 diabetes (UKPDS 33)”, Lancet 352:837-853, 1998).

Present blood glucose monitoring techniques based on blood collection by fingersticking and subsequent glucose determination with single use test elements (i.e., test strips) cannot be applied frequently enough to detect the early stages of blood glucose excursions (see Gough, D A, Kreutz-Delgado, K and Bremer, T M “Frequency characterization of blood glucose dynamics”, Ann Biomed Eng 31:91-97, 2003). Thus, even highly motivated patients who carefully perform frequent fingerstick measurements may miss substantial fluctuation in glucose levels, particularly episodes of nocturnal hypoglycemia (see Boland, E, Monsod, T, Delucia, M, Brandt, C A, Fernando, S and Tamborlane, W V “Limitations of conventional methods of self-monitoring of blood glucose: lessons learned from 3 days of continuous glucose sensing in pediatric patients with type 1 diabetes”, Diabetes Care 24:1858-1862, 2001).

In the following, intermittent blood glucose monitors will be described.

Currently a blood glucose testing system widely used by diabetic patients consists of a test strip and a measuring instrument. For analyzing a blood sample, a test strip is manually removed from a separate storage container and the rear or contact end of the test strip is inserted into the test strip holder of the instrument. After pricking the finger with a lancet, the testing end of the test strip is placed into the blood that has accumulated on the patient's finger. Due to capillary action, the blood is drawn through a capillary channel to the reagent materials contained in the test strip. The reagent materials then chemically react with the glucose drawn into the test strip to cause a detectable signal. The test strip is then disposed of after use (see Newman, J D and Turner, A P F “Home blood glucose biosensors: a commercial perspective”, Biosensors and Bioelectronics 20:2435-2453, 2005).

In the following, a multiple test strip device will be described.

Recently, several blood glucose testing systems have been brought to market in which the measuring instrument is adapted to receive a magazine containing a multitude of single-use test strips. The test strips are accommodated in slots or cavities of the magazine. To perform a blood glucose measurement, one of the test strips is mechanically removed from the magazine and positioned in the test strip holder with the testing end of the test strip projecting out from the instrument. When in this testing position, the testing end of test strip can be placed into the blood being analyzed. After the blood has been analyzed, the used test strip is ejected from the instrument (see Newman, J D and Turner, A P F “Home blood glucose biosensors: a commercial perspective”, Biosensors and Bioelectronics 20:2435-2453, 2005).

In the following, continuous glucose monitors will be described.

There has been a significant effort put forth by the research community toward the development of painless and continuous methods for monitoring blood glucose levels (see Gough, D A, and Armour, I C “Development of the implantable glucose sensor. What are the prospects and why is it taking so long?”, Diabetes 44:1005-9, 1995; Klonoff, D C “Current, emerging, and future trends in metabolic monitoring”, Diabetes Technol Ther 4:583-8, 2002; Kerner, W “Implantable glucose sensors: Present status and future developments”, Exp Clin Endocrinol Diabetes 109 (Suppl 2):S341-S346, 2001; Klonoff, D C, “Continuous glucose monitoring”, Diabetes Care 28:1231-9, 2005). Because of the potential risks associated with continuous blood glucose monitoring (e.g., infection, bleeding, clotting, sensor fouling), most of the studies in the recent past have focused on the interstitial fluid (ISF) of the peripheral tissues as an alternative site for the continuous monitoring of glucose levels (see Klonoff, D C “Current, emerging, and future trends in metabolic monitoring”, Diabetes Technol Ther 4:583-8, 2002; Kerner, W “Implantable glucose sensors: Present status and future developments”, Exp Clin Endocrinol Diabetes 109 (Suppl 2):S341-S346, 2001; Klonoff, D C “Continuous glucose monitoring”, Diabetes Care 28:1231-9, 2005.). The approaches for measuring ISF glucose levels thus far proposed fall into two general categories: ex vivo and in vivo approaches.

In the in vivo approach, a glucose sensor is inserted directly into the subcutaneous tissue. Commercially available technologies based on this approach are the Guardian RT System (Medtronic MiniMed Inc., Northridge, Calif.) and the DexCom STS Continuous Glucose Monitoring System (DexCom, San Diego, Calif.). These devices consist of an enzyme-tipped catheter sensor that is inserted into the subcutaneous tissue and wired to a transmitter worn externally. The transmitter sends information to a pager-sized monitor (see Mastrototaro, J J “The MiniMed continuous glucose monitoring system”, Diabetes Technol Ther 2 (Suppl 1):S13-S18, 2000; or http://www.dexcom.com). Because of stability problems, these available sensor techniques require rigorous calibration with blood glucose measurements obtained by fingerstick testing and are, therefore, indicated for use as an adjunctive device to complement, not replace, information obtained from standard blood glucose testing devices.

In the ex vivo approach, ISF is extracted from peripheral tissues and then transported to a glucose sensor that is located outside the body. This approach has been the basis of commercially available devices, which measure glucose from ISF that has been extracted either by means of a microdialysis catheter (see Maran, A, Crepaldi, C, Tiengo, A, Grassi, G, Vitali, E, Pagano, G, Bistoni, S, Calabrese, G, Santeusanio, F, Leonetti, F, Ribaudo, M, Di Mario, U, Annuzzi, G, Genovese, S, Riccardi, G, Previti, M, Cucinotta, G, Giorgino, F, Bellomo, A, Giorgino, R, Poscia, A and Varalli, M “Continuous subcutaneous glucose monitoring in diabetic patients: a multicenter analysis”, Diabetes Care 25:347-352, 2002) or a process denoted as “reverse iontophoresis” (see Tamada, J A, Bohannon, N J and Potts, R O “Measurement of glucose in diabetic subjects using noninvasive transdermal extraction”, Nat Med 1:1198-1201, 1995). The latter device is worn on the wrist or arm, and samples ISF that is drawn through the skin into an electrolyte by applying an electric field between two electrodes placed on the skin (i.e., reversed iontophoresis). The electrodes are coupled to the skin by the electrolyte. Glucose can then be measured using a glucose sensor in contact with the electrolyte.

Other systems based on the ex vivo approach are under development (see Kerner, W “Implantable glucose sensors: Present status and future developments”, Exp Clin Endocrinol Diabetes 109 (Suppl 2):S341-S346, 2001; Klonoff, D C “Continuous glucose monitoring”, Diabetes Care 28:1231-9, 2005).

U.S. Pat. No. 5,097,834 discloses that, in order to determine at least one parameter of interest in a living organism, a perfusion fluid is directly introduced in the tissues. After its partial balancing by the tissue parameter of interest, the perfusion fluid is collected and analyzed for the parameter of interest, as well as for endogenous or exogenous marker properties indicative of the degree of interaction between the perfusion fluid and the tissue, in such a way that the parameter of interest can be determined with the help of such characteristic properties.

U.S. Pat. No. 6,770,030 discloses a device for in-vivo measurements of quantities in living organisms that comprises a catheter-like tube receiving in a removable manner a setting needle provided for inserting the tube in the organism, with at least one aperture being provided in the wall of the tube, and a sensor for detecting the quantity to be measured in the interior of the tube. The sensor is mounted on a separate oblong carrier whose cross sectional dimensions are smaller than those of the interior of the tube. After the setting needle has been pulled out of the tube, the sensor can be inserted in said tube.

EP 1,166,808 discloses an injection needle unit usable in a portable automatic syringe device, the injection needle unit comprising a feeding tube, an “L”-shaped injection needle member connected to one end of the feeding tube, a connector connected to the other end of the feeding tube, and a depressing member integrally formed with the injection needle member in such a fashion that the injection needle protrudes perpendicularly from the depressing member, the depressing member being depressed against the skin of a user upon penetrating the injection needle member into the subcutaneous tissue of the user. The injection needle unit comprises a glucose sensor attached to the injection needle and adapted to penetrate the body of the user when the injection needle penetrates the body of the user. The glucose sensor comprises an electrode wire wound around an injection needle in the form of a core, an insulating layer coated over the injection needle to insulate the injection needle from the electrode wire, and an enzyme member fitted around a portion of the injection needle adjacent to the injection tip while being insulated from the electrode wire. The enzyme member and the electrode wire penetrate the body of the user when the injection needle penetrates the body of the user, and leads connected to the enzyme member and the electrode wire, respectively, to electrically connect the enzyme member and the electrode wire to a voltage sensing means included in the automatic syringe device.

U.S. Pat. No. 5,243,982 discloses a device for determining the concentration of at least one substance in organic tissue including a subcutaneous needle for insertion into the tissue and a pumping and suction unit which is controlled by a microprocessor and is used for delivering a perfusion fluid and draining it after its partial equilibration with the tissue. The device further includes a sensing unit connected to the microprocessor for determining the concentration of the substance to be analyzed and one or more marker variables. The perfusion fluid, a calibrating solution and at least one drug are provided in replaceable metering ampoules sealed with membranes, whose metering plungers can be actuated by a drive unit controlled by the microprocessor. The metering ampoules communicate with a master capillary leading from the pumping and suction unit to the subcutaneous needle. Communication is established via capillary tubes piercing the membranes.

U.S. Pat. No. 5,193,545 discloses a device designed for determining one or more medical variables in living organisms, comprising a part to be inserted into the tissue of the organism, which has an exchange channel with openings into the tissue. The device comprises a delivery unit connected with the exchange channel for the delivery of a perfusion fluid and its drainage after partial equilibration with the medical variable or quantity to be measured, i.e., preferably glucose concentration, and comparatively constant endogenous or exogenous marker variables. The device further comprises an analyzing unit for measuring the medical variable and one marker variable, and an evaluation unit for determining the actual concentration of the medical variable with the use of the marker variables.

U.S. Pat. No. 6,458,118 discloses an implantable drug delivery device comprising a microencapsulated drug contained within at least one capsule, a carrier fluid that will dissolve the drug when freed from the capsule, a drug releaser for freeing the microencapsulated drug from the capsule, a reservoir in which the carrier fluid dissolves the drug, and an electromechanical pump to convey the dissolved drug to a catheter, through which the drug is delivered to a target site within a patient.

DE 197 56 872 discloses a device for giving a transfusion and/or perfusion to a patient comprising one or several sensors for measuring real values of one or several patient-specific parameters; control means in communication with the sensor(s), and a transfusion and/or perfusion device containing the transfusion and/or perfusion solution to be given and in communication with the control means. The control means control the amount of transfusion and/or perfusion solution to be supplied by means of the transfusion and/or perfusion device depending on the sensed real values. The control means comprise an expert system for processing incoming real values and on the basis of which control is carried out.

It is an object of the invention to allow for an efficient determination of a value of a physiological parameter.

In order to achieve the object defined above, a device and a method for determining a value of a physiological parameter of a body fluid of a body under investigation according to the independent claims are provided.

According to an exemplary embodiment of the invention, a device for determining a value of a physiological parameter of a body fluid of a body under investigation is provided, the device comprising a fluid reservoir for containing a perfusate fluid, a body interface insertable into the body under investigation for a fluid communication between the fluid reservoir and the body fluid, a fluid conduit between the fluid reservoir and the body interface, a bidirectional fluid transport unit for transporting the perfusate fluid from the fluid reservoir through the fluid conduit and the body interface into the body under investigation and for subsequently transporting a mixture of the perfusate fluid and the body fluid from the body under investigation through the body interface into the fluid conduit, and a sensor for sensing the value of the physiological parameter based on an analysis of the mixture of the perfusate fluid and the body fluid.

According to another exemplary embodiment of the invention, a method of determining a value of a physiological parameter of a body fluid of a body under investigation is provided, the method comprising inserting a body interface into the body under investigation for enabling a fluid communication between the body fluid and a fluid reservoir containing a perfusate fluid, transporting, by a bidirectional fluid transport unit, the perfusate fluid from the fluid reservoir through a fluid conduit and the body interface into the body under investigation and subsequently transporting a mixture of the perfusate fluid and the body fluid from the body under investigation through the body interface into the fluid conduit, and sensing the value of the physiological parameter based on an analysis of the mixture of the perfusate fluid and the body fluid.

According to still another exemplary embodiment of the invention, a device for determining a value of a physiological parameter of a body fluid of a body under investigation is provided, the device comprising a fluid reservoir for containing a perfusate fluid, a body interface insertable into the body under investigation for a fluid communication between the fluid reservoir and the body fluid, a fluid conduit between the fluid reservoir and the body interface, a fluid transport unit for transporting the perfusate fluid from the fluid reservoir through the fluid conduit and the body interface into the body under investigation, a sensor positioned at the body interface and adapted for sensing the value of the physiological parameter based on an analysis of the body fluid, particularly based on an analysis of the mixture of the perfusate fluid and the body fluid, and a calibration unit positioned at the body interface and adapted for determining a mixture degree of the perfusate and the body fluid.

The term “body under investigation” may particularly denote any human being, any animal, and any plant (any organism). It may be a living body so that living tissue may be investigated.

The term “physiological parameter” may particularly denote any parameter which is related to the physiology of a living organism, for instance the metabolism, etc. Such a physiological parameter may include the concentration of an exogenous or endogenous marker, a protein concentration, etc. The physiological parameter to be measured may preferably be a glucose concentration in the body (particularly a tissue glucose concentration in the body), but may alternatively or additionally also be a lactate concentration in the body, an oxygen concentration in the body, an ion concentration (such as hydrogen-ion concentration, i.e., pH) in the body, a cholesterol concentration in the body, a quantity of bacteria in the body, a quantity of viruses in the body, or a medication concentration in the body. In this way, the measurement of such a physiological parameter or another physiological parameter may provide information about a current state of the body and thus be used as a meaningful decision criterion as to whether and to what extent a physiologically active substance is to be supplied to the body locally or systemically.

The term “fluid” may include any substance in this context which at least partially contains components in the liquid phase. Of course, solid and/or gaseous components may also be contained or dissolved in such a fluid and may even make up the predominant component of the fluid. Thus, the penetrated part of the body may be particularly subcutaneous tissue, an organ, a vein, an artery, and a blood vessel. An intravenous application of the device is possible. Also applications in the skin are possible, for instance by cutaneous ultrafiltration or microperfusion. The fluid which is removable from the body may be tissue liquid. Alternatively or additionally, the fluid may, however, also be blood, lymph, spinal fluid, urine, or other tissue.

The term “perfusion fluid” may particularly denote any carrier liquid in which the physiologically active substance may be optionally included, dissolved or diluted so as to obtain a solution of a physiologically active substance in a desired concentration. It is common practice to provide physiologically active substances, like insulin, in a liquid perfusate solution in which the physiologically active substances is contained. Particularly, the skilled person knows many examples for perfusion fluids which are appropriate for containing insulin or other physiologically active substances. A perfusion fluid may also comprise or consist of a neutral solution, a rinse fluid, or a cleaning fluid.

The term “physiologically active substance” may particularly denote any substance which may have an effect on the physiology of a living organism, for instance a medication, a drug, etc. The physiologically active substance may be a medication, particularly a glucose-regulating medication such as insulin, insulin analogues, glucagon, catecholamines, cortisol, or growth hormone. In this way, the supply of the physiologically active substance to the body may selectively and effectively influence the glucose level in the body or in specific parts of the body (e.g., tissue, blood). However, the physiologically active substance may also be any other arbitrary substance (such as aldosterone, bicarbonate, oxygen, phosphate) which has a functional influence on the body or organism. This particularly also includes any type of medicine, vitamin, carrier substances (such as artificial oxygen carriers), etc.

According to an exemplary embodiment of the present invention, is may be made possible to efficiently combine insulin delivery and glucose determination techniques to provide a system which allows a proper mimicking of the glucose-regulating function of a healthy pancreas even over a long period of time and may thus be used in the context with type 1 diabetes. For high acceptance, the system may be constructed small and compact and may be able to be worn comfortably on the body.

According to an exemplary embodiment of the invention, a physiological parameter may be determined by injecting a drop of a perfusate fluid through a body interface into a body under investigation. After this drop has been injected into the body, components of this fluid may equilibrate with surrounding body fluid. For example, a glucose concentration of surrounding body fluid may equilibrate with the perfusate fluid so that the glucose concentration in the perfusate fluid may be a fingerprint or an indication for the glucose concentration in the body fluid. After such a sufficient equilibration or mixing between the perfusion fluid and components of the surrounding body fluid has occurred, a pumping direction of the pump which has previously pumped the perfusion fluid from the device into the body may be simply reversed so that the equilibrated or modified perfusion fluid is sucked again into the device. In other words, the pumping direction of the bidirectional pump may be inverted to transport back the perfusion fluid into the device. The glucose concentration in the modified perfusion fluid may be measured so as to obtain a reliable value for this parameter.

This procedure can be combined with a supply of a physiologically active substance. For example, insulin or any other physiologically active substance may be dissolved in the perfusion fluid so that infusing the perfusion fluid into the body for the purpose of measuring the physiological parameter may simultaneously feed a medication inside the body. Therefore, the sensing and supply procedures may be advantageously combined. The amount of the fed physiologically active substance may be varied by varying a flow rate of the pump.

The use of (for instance a single) bidirectional pump in combination with (for instance a single) fluid conduit connecting the fluid reservoir for the perfusate fluid with the body interface to be inserted into the human body is a very simple, but very efficient way of detecting a physiological parameter and/or supplying of a physiologically active substance.

For example, a fluid drop may be deposited as a probe in the body where the probe may be mixed with body fluid. Then, the modified probe may be led back into the device for analysis. Such a procedure may pump the probe in one direction and pump it back into the device in an opposite direction. This may be realized using a peristaltic pump which has a first forward operation mode and a second backward operation mode. By using the probe multiple times, a waste may be dispensable, since an analyzed fluid can be led back afterwards.

Separate fingersticking for measuring a glucose concentration and separate insulin supply may become dispensible.

Such a system may not only measure the value of the physiological parameter but may combine this with an endogenous and/or exogenous marker measurement, such as the conductivity measurement, thereby allowing to determine whether a mixture or equilibration between the perfusion fluid and the body fluid is sufficient so as to get a meaningful result for the value of the physiological parameter.

Thus, according to an exemplary embodiment of the invention, a continuous insulin supply may be performed by means of a perfusate fluid as a carrier in which insulin is dissolved. After this insulin drop has been supplied to the body, a pumping direction of the pump may be inverted to pump back the perfusate into the device in order to monitor whether an endogenous marker indicates a sufficient mixture between body fluid and perfusion fluid. Therefore, through the same fluid conduit through which the perfusate has previously been supplied from the fluid reservoir to the body interface, the modified perfusate fluid may flow back into the device for analysis. A controller may decide whether the mixture between the perfusion fluid and the body fluid is sufficient, and if such a comparison yields a positive result, the sensor result may be accepted.

Particularly, it may be sufficient to have a single fluid conduit between the therapy device and the body. A perfusate may be infused into a body for a glucose measurement, and after that a mixture of the perfusate and the body fluid can be used for measuring.

In order to obtain a sufficiently high recovery rate, it is possible to switch the bidirectional pump several times (for instance two, three, four, five times), so as to obtain an improved mixture between perfusion fluid and body fluid. Additionally or alternatively, one or more perforations may be provided in a wall of the body interface which may improve the mixture characteristics or recovery rate as well. For instance, such holes may have a size of 0.3 mm. For example, 28 such holes may be provided in a body interface.

According to an exemplary embodiment of the invention, the spatial extension of the injected fluid at the position at which the physiologically active substance (for instance insulin) is inserted or introduced in the body and the position at which the value of the physiological parameter (for instance a glucose level) is measured, may be of interest.

The perfusate fluid introduced in the body of course has a non-zero volume or, in other words, a three-dimensional extension. At a border area between this fluid volume (formed by the injected perfusion fluid and the physiologically active substance being optionally dissolved therein) and the surrounding or adjacent body fluid (particularly tissue fluid or interstitial fluid), it is believed that an exchange of molecules may occur between these two liquid phases (for instance by mechanisms like diffusion, osmosis-like phenomena, convectional mixing, etc.). Particularly, it is believed that insulin as the physiologically active substance diffuses into the body fluid, and glucose diffuses from the tissue fluid into the “drop” of injected perfusion fluid. These phenomena are believed to occur due to a temporary non-equilibrium or concentration gradient between the area of influence of the delivered insulin (that is the phase of the perfusion fluid including the physiologically active substance) and the tissue fluid. The two phases are selectively left uninfluenced for a predetermined waiting time or contact time or interaction time. The time interval may depend on a specific application and may be estimated by carrying out routine experiments, by measuring the concentration of endogenous and/or exogenous markers, by applying theoretical or empiric models, or may be known from experience. The time interval may be in the order of magnitude between seconds and hours, particularly in the order of magnitudes of minutes. Depending on the duration of the time interval, a partial or an essentially complete equilibrium of the concentrations of the physiologically active substance (insulin) and the substance to be measured for estimating the physiological parameter (glucose) is obtained. In this (partial) equilibration state, the concentration of the glucose may be measured in the modified perfusate liquid. This may be performed particularly by one of the two following procedures. According to a first procedure, the modified perfusate can be removed (partially or essentially) completely from the body and may then be measured with an externally positioned sensor (for instance located in a fluid conduit connecting fluid reservoir and body interface). According to a second procedure, the glucose level in the modified perfusate can be measured by a sensor located adjacent to or functionally coupled with a modified perfusate in the body (for instance located at or in an exterior wall of the body interface).

According to an exemplary embodiment, it may be advantageous that a bidirectional pump transports the perfusate into the body and the modified perfusate through the same conduit back to the device, which may make the device small, efficient and cheap.

Next, further exemplary embodiments of the devices will be explained. However, these embodiments also apply to the method.

The device may be adapted for supplying a physiologically active substance to the body under investigation. Thus, not only a physiological parameter may be determined using the perfusate fluid, but it is also possible that a physiologically active substance may be supplied to the body using the perfusate fluid as a carrier.

The device may comprise a control unit adapted for controlling the bidirectional fluid transport unit for transporting the perfusate fluid from the fluid reservoir through the fluid conduit and the body interface into the body under investigation, and for subsequently transporting the mixture of the perfusate fluid and the body fluid from the body under investigation through the body interface into the fluid conduit. Such a control unit (for instance a microprocessor or a CPU, central processing unit) may centrally control operation of two or more of the individual components of the device. The control unit may generate control signals to control or regulate a pump, particularly to adjust a pumping direction of the pump. By taking this measure, a single fluid conduit may be used to transport fluid selectively in a forward and a backward direction, to first insert the perfusate fluid as a probe into the body, and after equilibration with the body fluid, suck this fluid back into the device again for analysis of the physiological parameter.

It is also possible that the control unit controls the bidirectional fluid transport unit for transporting the mixture of the perfusate fluid and the body fluid from the body under investigation through the body interface into the fluid conduit only upon detecting that the perfusate fluid and the body fluid have been mixed sufficiently to exceed a predetermined mixture degree. Only when a proper mixture between the perfusate probe and the body fluid has been verified, a meaningful sensing result may be obtained. For this purpose, the mixture may be detected, for instance by a conductivity sensor. If the conductivity sensor indicates that a proper mixture has occurred, this may be used as a trigger for the control unit to control the bidirectional pump into another operation state.

The control unit may be adapted for controlling the bidirectional fluid transport unit to switch a plurality of times between an operation state in which the perfusate fluid is transported from the body interface into the body under investigation, and an operation state in which the mixture of the perfusate fluid and the body fluid is transported from the body under investigation into the body interface. By repeating a pumping and sucking procedure of the perfusate probe several times, the substance equilibration (or recovery rate) between the oscillating perfusate probe and the surrounding body tissue may be promoted. Additionally or alternatively, an end of the body interface may comprise one or more perforations which may further promote the equilibration. Therefore, by taking one or both of these measures, a recovery rate may be improved.

The device may comprise exactly one fluid conduit between the fluid reservoir and the body interface. Thus, a single fluid conduit may be sufficient between the fluid reservoir and the body interface, more particularly between the body and an inlet of the device, since the bidirectional transport of the pump may allow to use both flowing directions of the fluid conduit. This may allow to manufacture the device in a small size.

The device may comprise a bifurcation-free fluid conduit between the fluid reservoir and the body interface. The term “bifurcation-free” may denote that no bifurcations are provided in the fluid conduit but that a fluid entering one end of the fluid conduit is transported without bifurcations or branches to another end of the fluid conduit. This may allow to manufacture the device in small size and with low effort.

The sensor may be positioned within the fluid conduit, that is between the body interface and the fluid container. Therefore, when the modified perfusate probe is sucked back into the device, a sensor may be automatically brought in fluid communication with the modified perfusate. This may allow for an efficient detection.

Alternatively, the sensor may be positioned at/in an interior wall of the body interface. The body interface may have an internal tubular section through which the fluid may be guided during analysis. When the fluid is sucked back into this internal tubular section of the body interface, the sensor will be automatically brought in contact with the modified perfusate probe so as to be able to perform a proper sensing.

Alternatively, the sensor may be positioned at/in an exterior wall of the body interface. By sucking back at least a portion of the perfusate fluid into the device may then allow the exteriorly positioned sensor to be brought in functional contact with the modified perfusate probe or with the (essentially pure) body fluid.

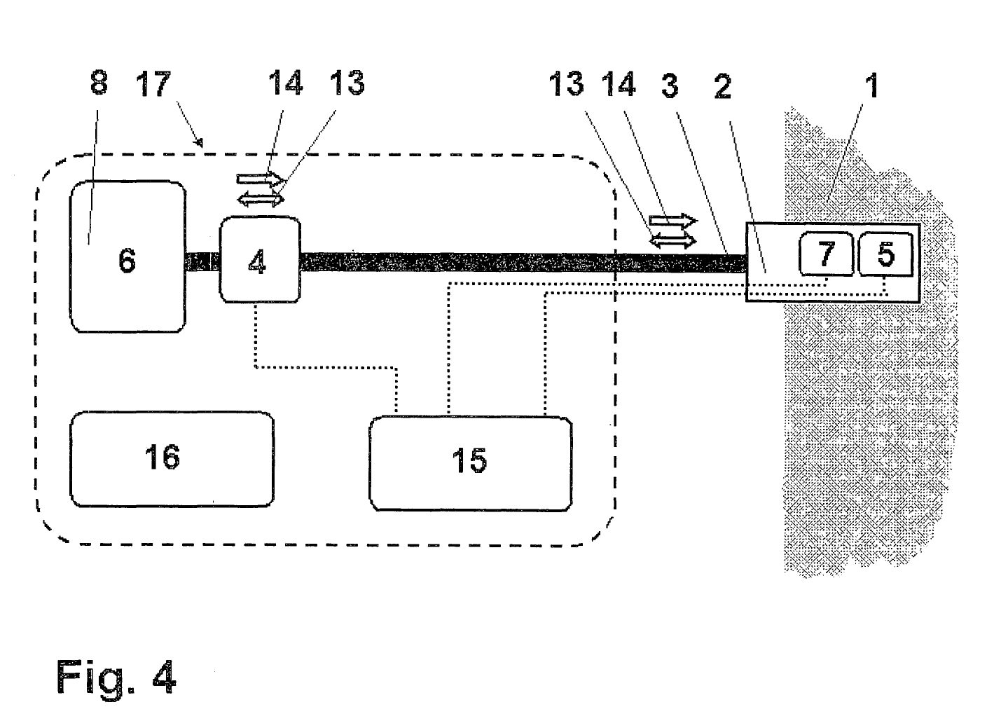

Alternatively, it is possible to simply inject the perfusate comprising the physiologically active substance (like insulin) into the body and to measure the physiological parameter (like glucose level) without the need to suck back the liquid (see arrow 14 in FIG. 4). Since a (back-)flow of the perfusate between tissue and surface of the body interface is possible, the environment around the sensor can be “diluted”. This dilution or equilibration or mixture degree can be measured by a marker, and the glucose measurement may be corrected accordingly. It is also possible to stop the infusion of the perfusate, and to wait until equilibration. Afterwards the glucose can be reliably measured. Sucking back the perfusion fluid in the body interface would then be provided for accelerating the equilibration procedure. (see arrow 13 in FIG. 4).

The body interface may comprise a wall having a plurality of fluid-exchange perforations. Such perforations, for instance at least two perforations, which may have a size of between 0.1 mm and 0.8 mm, more particularly essentially 0.3 mm, may improve the fluid exchange at the position of the body interface, thereby allowing an accurate detection.

The device may comprise a single bidirectional fluid transport unit. In other words, exactly one bidirectional fluid transport unit may be provided. Therefore, a single pump, particularly a single peristaltic pump which may be provided in a miniature format (for instance with a size in the dimension of 5 mm) may be sufficient. It is also possible to use a single fluid conduit for supplying the perfusate probe to the body, and vice versa. Particularly, the fluid transport direction of the bidirectional fluid transport unit may be invertible. In other words, the fluid may be selectively forced to flow along a first direction or along a second direction which may be opposite to the first direction.

A calibration unit may be provided in the device for determining a mixture degree indicative of a mixture ratio between the perfusate and the body fluid. For instance, a conductivity measurement may be a proper indicator whether the mixture between body fluid and perfusion fluid has been sufficient for a meaningful subsequent sensor analysis. The amount of the physiologically active substance (for instance insulin) to be supplied to the body may be dosed quantitatively on the basis of the value of the physiological parameter which has been sensed by the sensor (for instance the glucose level).

A mixture degree determined by the calibration may be taken as a basis for the control unit to decide whether a physiologically active substance shall be supplied to the body, and in which concentration or dose.

The control unit may be adapted to accept or reject to determine the amount of the physiologically active substance based on the mixture degree determined by the calibration unit. For instance, when the mixture degree is too small, the control unit may reject to use a sensor parameter for adjusting an amount of the physiologically active substance. If the mixture degree is sufficiently large, for instance exceeds a predetermined threshold value, the measurement may be accepted and the insulin dose may be selected in accordance with the sensor result. By taking this measure, the reliability of the system may be increased.

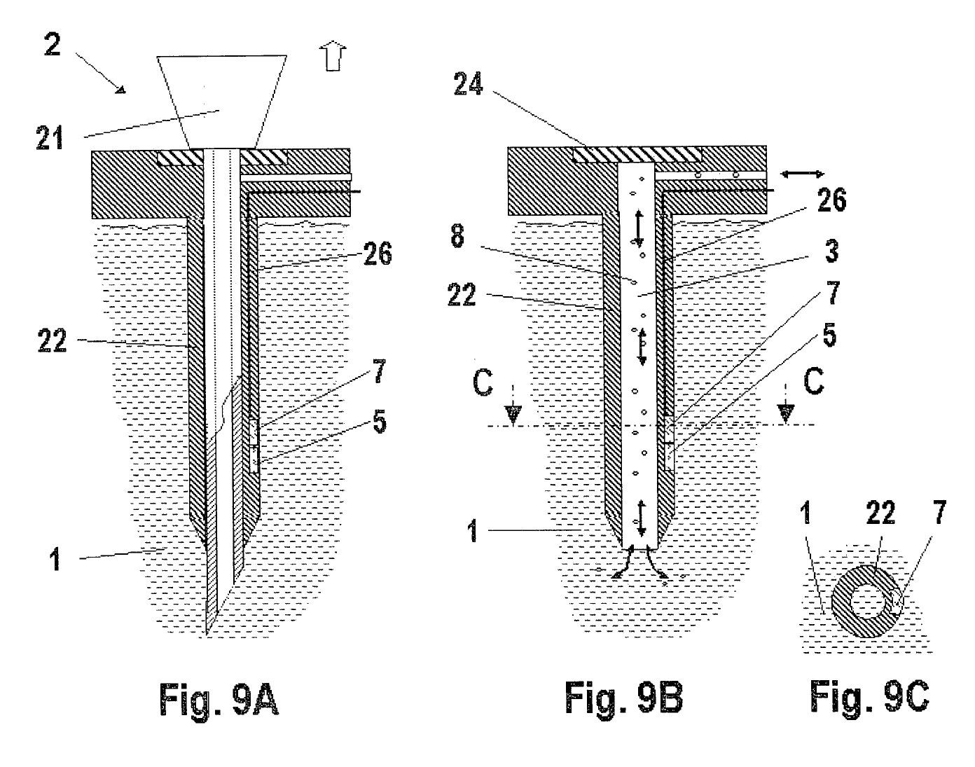

Due to a possible (back-)flow of the perfusate between the tissue and the catheter surface, the environment around the sensor can be “diluted”. This dilution or equilibration or mixture degree may be measured using a marker, and the glucose measurement may be corrected accordingly. It is also possible to stop or interrupt the perfusate infusion, to wait until an essentially completely equilibration has occurred, and to measure the glucose level then. The information with regard to the time at which the equilibration is essentially finished can be obtained from the marker sensor. Sucking back the perfusate fluid into the catheter can be performed for accelerating equilibration procedure (see arrows in FIG. 9B).

The body interface may be adapted to extract the mixture of the perfusate fluid and the body fluid from the body under investigation. In other words, the pump may be brought in a pumping direction in which the mixture is pumped back from the body into the device, for analysis.

Particularly, the sensor may be adapted to bring the extracted mixture of the perfusate fluid and the body fluid in functional contact with one or more test units, each of the test units being indicative of the present value of the physiological parameter when brought in functional contact with the extracted mixture of the perfusate fluid and the body fluid. For this purpose, a reception may be provided in the sensor into which a (for instance single-use) test unit may be inserted. For example, a glucose measuring strip may be inserted into the sensor via a human user. This may allow for an easy and simple measurement of the glucose level with a perfusion fluid which simultaneously may comprise the necessary insulin.

Beyond this, the device may be adapted for determining at least one of the physiological parameters selected from the group consisting of a glucose concentration, a lactate concentration, an oxygen concentration, an ion concentration, a cholesterol concentration, an amount of bacteria, an amount of virus, a drug concentration and a medicament concentration. In other words, the physiological parameter may be a concentration of a physiologically active substance in the body or at a spatial position inside the body. Thus, the physiological parameter may be any medically relevant parameter characterizing the body fluid and/or the state of the body from which the body fluid has been taken. Such information may be used to determine whether and to which amount it is necessary or not necessary to provide any medicament, perform any treatment, initiate any alarm, repeat measurement, or control any parameter.

For example, a patient suffering from diabetes may be the subject of the investigation, and the glucose concentration and/or an insulin concentration of such a person may be estimated.

The device may be adapted for determining the physiological parameter quasi-continuously over time or intermittently over time. In this respect, the term “continuously” (or quasi-continuously) may particularly denote that no or only small intervals are between points of time in which the value of the physiological parameter is monitored. By such an investigation, a time dependence of the physiological parameter may be derived from the investigations. Alternatively, it may be possible to determine the physiological parameter intermittently over time, that is to repeat the determination of the physiological parameter after expiry of a particular time interval. For instance, the glucose concentration of a patient may be determined every 15 minutes, every 30 minutes, or every 60 minutes.

The body interface may comprise at least one of the group consisting of a transcutaneous indwelling catheter interface, microdialysis interface, a microperfusion interface, an ultrafiltration interface, a porous tissue contactor interface, a iontophoresis reversed-iontophoresis interface, a suction-delivery technique interface using at least one microneedle, and a transdermal extraction-delivery interface using ultrasound and/or osmotic extraction buffer.

A flexible, transcutaneous indwelling cannula is disclosed in U.S. Pat. No. 5,257,980 to which explicit reference is made herewith. This type of body interface is introduced by the patient into the subcutaneous tissue with the aid of a metal needle and then may dwell for a long time in the tissue after removal of the metal needle.

The technique of “ultrafiltration” as such is disclosed in U.S. Pat. No. 5,002,054, to which explicit reference is made herewith with regard to examples as to how to perform ultrafiltration in the context of embodiments of the invention.

A “suction technique using microneedles” is disclosed as such in U.S. Pat. No. 6,689,100, to which explicit reference is made herewith with regard to examples as to how to perform this technique in the context of embodiments of the invention.

The technology of“transdermal extraction using ultrasound and/or osmotic extraction buffer” is disclosed as such in Kost, J, Mitragotri, S, Gabbay, R A, Pishko, M and Langer, R “Transdermal monitoring of glucose and other analytes using ultrasound”, Nat Med 6:347-350, 2000, and in Chuang, H, Taylor, E and Davison, T W “Clinical Evaluation of a continuous minimally invasive glucose flux sensor placed over ultrasonically permeated skin”, Diabetes Technol Ther 6:21-30, 2004, to which explicit reference is made herewith with regard to examples as to how to perform transdermal extraction using ultrasound and/or osmotic extraction buffer in the context of embodiments of the invention.

The technology of “reversed iontophoresis” is disclosed as such in Tamada, J A, Bohannon, N J and Potts, R O “Measurement of glucose in diabetic subjects using noninvasive transdermal extraction”, Nat Med 1:1198-1201, 1995, to which explicit reference is made herewith with regard to examples as to how to perform reversed iontophoresis in the context of embodiments of the invention. Such a device may be worn on the wrist or arm of a patient, and may sample interstitial fluid that is drawn through the skin and to an electrolyte by applying an electric field between two electrodes placed on the skin. The electrodes may be coupled to the skin by the electrolyte. Glucose or any other physiological parameter can then be measured using a glucose sensor in contact with the electrolyte.

The technique of “microdialysis” as such is disclosed in Schoemaker, M, Andreis, E, Roper, I, Kotulla, R, Lodwig, V, Obermaier, K, Stephan, P, Reuschling, W, Rutschmann, M, Schwaninger, R, Wittmann, U, Rinne, H, Kontschieder, H and Strohmeier, W “The SCGM1 System: subcutaneous continuous glucose monitoring based on microdialysis technique”, Diabetes Technol Ther 5:599-608, 2003, to which explicit reference is made herewith with regard to examples as to how to perform microdialysis in the context of the invention.

The technology of “microperfusion” as such is disclosed in Trajanoski, Z, Brunner, G A, Schaupp, L, Ellmerer, M, Wach, P, Pieber, T R, Kotanko, P and Skrabal, F “Open-flow microperfusion of subcutaneous adipose tissue for on-line continuous ex vivo measurement of glucose concentration”, Diabetes Care 20:1114-1121, 1997; Schaupp, L, Ellmerer, M, Brunner, G A, Wutte, A, Sendlhofer, G, Trajanoski, Z, Skrabal, F, Pieber, T R and Wach, P “Direct access to interstitial fluid in adipose tissue in humans by use of open-flow microperfusion”, Am J Physiol Endocrinol Metab 276:E401-E408, 1999; Regittnig, W, Ellmerer, M, Fauler, G, Sendlhofer, G, Trajanoski, Z, Leis, H., Schaupp, L, Wach, P and Pieber, T R “Assessment of transcapillary glucose exchange in human skeletal muscle and adipose tissue”, Am J Physiol Endocrinol Metab 285:241-51, 2003, to which explicit reference is made herewith with regard to examples as to how to perform microperfusion in the context of the invention.

The device may be adapted for determining the value of the physiological parameter of the body fluid of the group consisting of interstitial fluid, blood, lymph, cerebrospinal fluid, urine and tissue. The term interstitial fluid may, in the context of this application, particularly denote liquid located between the cells and tissue of the body. Particularly, interstitial fluid of the subcutaneous adipose tissue may be a good choice for a sample to determine the glucose concentration of a patient (for instance suffering from diabetes) in a gentle manner.

The sensor may comprise a reception for receiving an exchangeable test unit. Such an exchangeable test unit may be a glucose sensor which may be injected for a single use by a user to determine the actual glucose value. The result of this detection may then serve as a basis for determining which amount of glucose and/or insulin should be delivered to the body.

The sensor may comprise a multiple test unit device including a plurality of test units. Such a multiple test unit device may be realized as a replaceable or substitutable device. Such a multiple test unit device may be a disk-shaped magazine or a cylindrical magazine. Thus, a magazine may be a flat circular disk or a cylindrical drum. Test units (for instance test strips) may be accommodated in slots or cavities of such a magazine. To perform a measurement, one of the test units may be positioned so as to contact a body fluid sample to be investigated. When in this testing position, the testing end of the test strip can be placed into the body fluid sample being analyzed. After the body fluid sample has been analyzed, the used test strip may be ejected from the instrument or may be stored in a waste container.

Such a multiple test unit device may be designed in manner similar as disclosed in one of the documents U.S. Pat. No. 6,475,436, U.S. Pat. No. 5,510,266, U.S. Pat. No. 5,660,791, U.S. Pat. No. 5,489,414, U.S. Pat. No. 5,720,924, U.S. Pat. No. 5,863,800, U.S. Pat. No. 5,798,031, and may be manufactured in accordance with a method as disclosed in US 2005/0125162 or U.S. Pat. No. 5,407,554.

The multiple test unit may be a rotatable magazine. The rotation may be controlled by means of the control unit. Energy for the rotation may be provided by muscle force of a user or by an electric energy supply of the device, for instance a battery.

The plurality of test units may be test strips. Each of these test strips may have a particular portion which is provided to be brought in contact with the sample in order to initiate or promote a characteristic alteration of at least one property of this test strip region indicative of the current value of the physiological parameter to be monitored.

The sensor may be adapted for at least one of the group consisting of an optical detection, an electrical detection and a chemical detection.

An optical detection unit may determine information concerning the physiological parameter as a consequence of a modification of at least one optical property for a test unit after being brought in contact with the body fluid sample. For instance, the colour, the reflectivity, the absorption or a fluorescence property of such a test strip may be altered in a characteristic manner in dependence of the value of the physiological parameter.

Alternatively, the detection may be performed in an electrical manner, for instance using an effect like a modification of an ohmic resistance, a conductivity, a capacity, a magnetic parameter or the like of the test strip.

A chemical detection may be based on the fact that a test unit, after being brought in contact with the body fluid sample, may have modified chemical or biochemical characteristics, for instance a modified pH value, a modified concentration of a chemical substance, or the like.

For instance, a test unit may comprise glucose oxydase as sensor active substance. In the presence of glucose, glucose oxydase may be chemically modified so that an electrical voltage may be generated (which may be measured in the context of an electric chemical detection) and/or that a dye may be generated (which may be measured in the context of a photometric detection).

Reagent materials chemically react with the glucose brought in contact with the test strip to cause an electrical signal (so-called electrochemical measuring technique) or a colour change (so-called colorimetric measuring technique) in the test strip. Subsequently, the electrical signal or the colour change may be evaluated by the measuring instrument.

The control unit may be a microprocessor. Such a microprocessor may be a central processing unit (CPU). It may be manufactured as an integrated circuit (for instance monolithically integrated in a semiconductor substrate) and can be manufactured, for instance, in silicon technology. Such a microprocessor may have computational resources and may, for instance, have access to a memory device (for instance an EEPROM) for storing data. Such a control unit may further control the entire or a part of the functionality of the device and may receive control commands from a user interface.

Furthermore, the control unit may initiate output of information to a user interface, for instance provide the result of the monitoring in a graphical manner (in real time or as a retrospective). For example, the microprocessor may generate output information indicative of the current glucose value of the body under investigation.

The bidirectional fluid transport unit may be a pump, particularly a peristaltic pump. Such a pump may be a micropump or a miniature pump.

The device may comprise an energy supply adapted to supply at least a part of the device with energy. For instance, units like the control unit, a memory device, a pump, an optical detection unit, or the like may require electrical energy which may be provided by such an energy supply unit. The energy supply may be provided to be rechargeable or replaceable.

For instance, the energy supply unit may be a battery, a fuel cell, or a solar cell. Realizing the energy supply unit by a battery, a single use battery or a rechargeable accumulator may be used. When using a solar cell, no separate energy supply unit is required and light in the environment may be used as a source of energy for operating the device.

The fluid reservoir may be adapted to hold a medication, particularly a glucose regulating substance, more particularly at least one of the group consisting of insulin, glucagon, aldosterone, and bi-carbonate.

Thus, the device may also control injection, infusion or insertion of any material into the body under investigation, for instance buffers, test fluids, medications or the like.

The reservoir unit may be adapted to hold a glucose regulating substance (that is particularly any substance which may have an influence on the glucose level in an organism), like insulin, an insulin-like growth hormone, adrenaline or the like.

Particularly, the reservoir unit may be adapted to hold a medication as the infusion fluid. Therefore, as a consequence of the determination of the value of physiological parameter, the evaluation of the device may decide that it is necessary to infuse a particular amount of medication into the patient's body. For instance, when it has been detected that the glucose level has become too high, insulin may be infused in the body of the person. For instance, when it has been detected that the glucose level has become too low, glucose may be infused in the body of the person.

The reservoir unit may be adapted to hold insulin as the infusion fluid. However, the reservoir unit may, additionally or alternatively, be adapted to hold aldosterone or bicarbonate or any other physiologically active substance as the infusion fluid.

For instance, the hormone aldosterone may be injected into a body under investigation, as a reaction to the determination of corresponding values of a physiological parameter like an ion concentration, for instance the Potassium concentration. Aldosterone regulates the electrolyte and water concentrations in the human body. It increases resorption of Sodium ions from the kidney, thus increasing the Sodium level in the blood. Secretion of Potassium ions and water ions is promoted. Consequently, the Potassium level in the blood is reduced. Simultaneously, water is retained. Therefore, aldosterone also has an influence on the regulation of the blood volume and the blood pressure.

Another example for a physiological parameter and an assigned medication is the lactate or oxygen concentration in blood (as physiological parameter) and a bicarbonate infusion (as the medication). In case of blood circulation distortions in tissue, the oxygen concentration may be reduced and the lactate concentration may be increased due to a less efficient glucose metabolism. If the presence of such a scenario is detected by measuring the lactate or oxygen concentration in blood as the physiological parameter, substances like bicarbonate or oxygen may be infused in order to locally improve blood circulation.

The fluid reservoir may also be adapted to hold calibrating substances for calibration of the sensor for measuring the physiological parameter and/or calibration of the endogenous/exogenous marker sensor.

The aspects defined above and further aspects of the invention are apparent from the examples of embodiment to be described hereinafter and are explained with reference to these examples of embodiment.

The invention will be described in more detail hereinafter with reference to examples of embodiment but to which the invention is not limited.

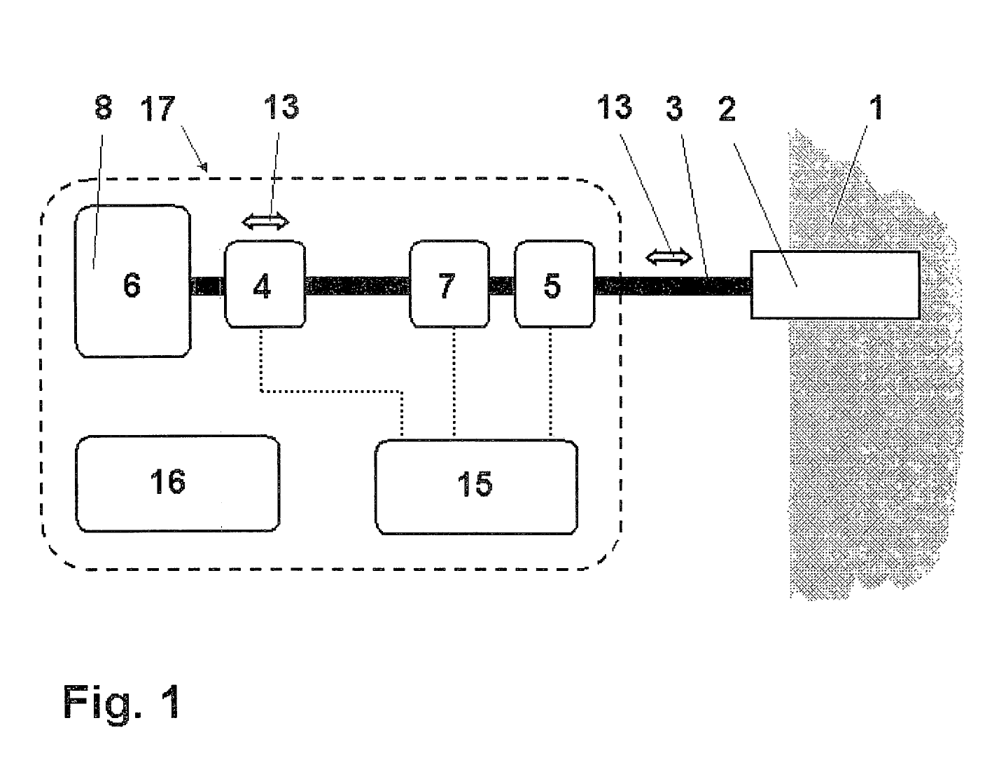

FIG. 1 to FIG. 4 show devices for determining a value of a physiological parameter of a body fluid of a body under investigation and for supplying a physiologically active substance to the body under investigation according to exemplary embodiments of the invention.

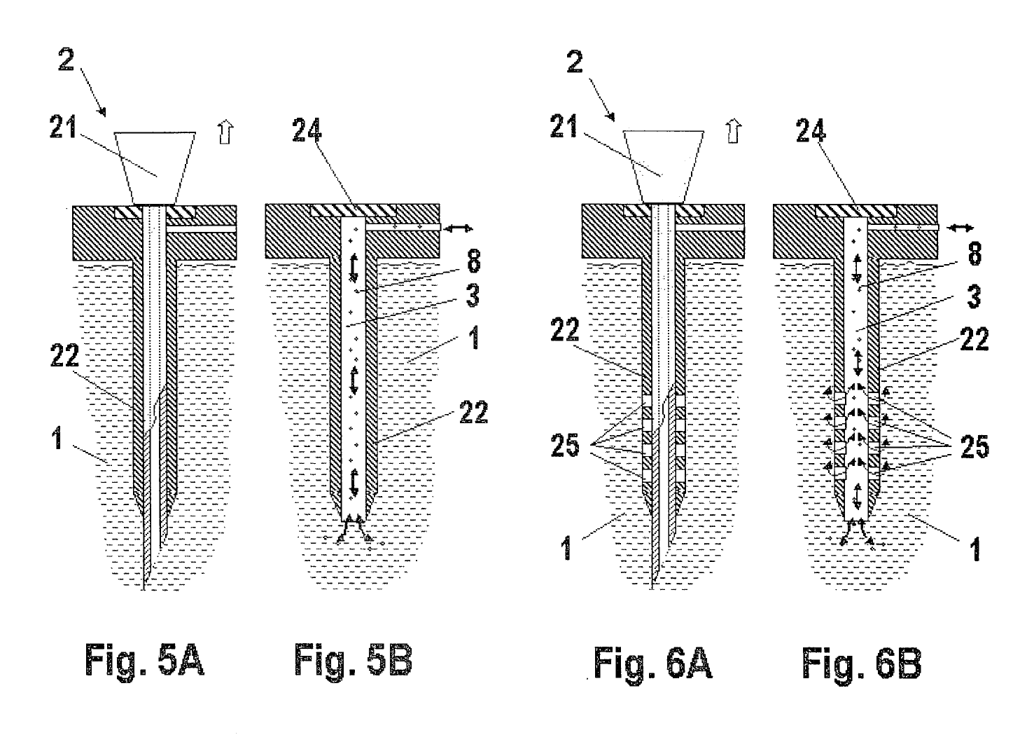

FIG. 5A to FIG. 9C show body interfaces implementable in devices shown in FIG. 1 to FIG. 4 according to exemplary embodiments of the invention.

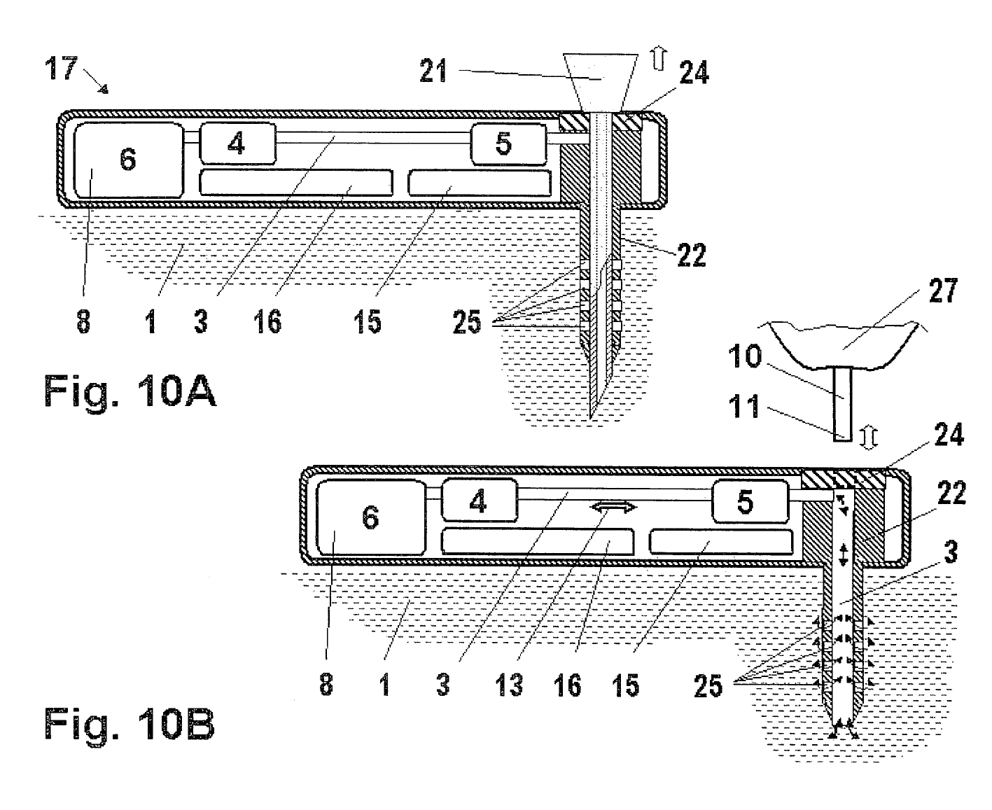

FIG. 10A and FIG. 10B show devices for determining a value of a physiological parameter of a body fluid of a body under investigation and for supplying a physiologically active substance to the body under investigation according to exemplary embodiments of the invention.

The illustration in the drawing is schematically. In different drawings, similar or identical elements are provided with the same reference signs.

In the following, referring to FIG. 1, a device 17 for determining a glucose level of blood or an interstitial fluid of a human body 1 under investigation according to an exemplary embodiment of the invention will be explained.

The device 17 shown in FIG. 1 comprises a fluid reservoir 6 comprising a perfusate fluid including insulin 8. Furthermore, the device 17 of FIG. 1 comprises a body interface 2 insertable into the body 1 under investigation for a fluid communication between the fluid reservoir 6 and the body fluid. Moreover, a fluid conduit 3 is provided between the fluid reservoir 6 and the body interface 2. A bidirectional (see double arrow 13) pump 4 is provided for transporting the perfusate fluid from the fluid reservoir 6 through the fluid conduit 3 and the body interface 2 into the body 1 under investigation, and for subsequently transporting a mixture of the perfusate fluid and the body fluid from the body 1 under investigation through the body interface 2 into the fluid conduit 3.

Moreover, a glucose sensor 7 is provided for sensing the glucose value based on an analysis of the mixture of the perfusate fluid and the body fluid.

By providing also insulin 8 as a physiologically active substance in the perfusion fluid container 6, a simultaneous supply of insulin to the body 1 and an analysis of the same fluid drop suctioned into the conduit 3 is made possible.

A control unit 15 is shown and adapted for controlling the bidirectional pump 4 for transporting the perfusate fluid from the fluid reservoir 6 through the fluid conduit 3 and the body interface 2 into the body 1 under investigation and for subsequently transporting the mixture of the perfusate fluid and the body fluid from the body 1 under investigation through the body interface 2 into the fluid conduit 3.

Furthermore, a calibrator unit 5 is provided measuring conductivity (or more generally an endogenous or exogenous marker) of the mixture and being connected for data communication with the control unit 15. The conductivity value may be supplied to the controller 15. Based on the conductivity value, the controller 15 may decide whether a sufficient mixture between the body fluid and the perfusate has occurred. If this is the case, a sensor result of a sensor 7 (being connected for data communication with the control unit 15) is used by the controller 15 to determine an amount of insulin to be delivered to the living human body 1. The sensor 7 is positioned within the fluid conduit 3.

The control unit 15 may control the pumping direction of the pump 4. In a first operation mode, the fluid is pumped, according to FIG. 1, from the left-hand side to the right-hand side. In a second operation state, according to FIG. 1, the fluid is pumped from the right-hand side to the left-hand side.

As shown in FIG. 1, exactly one bifurcation-free fluid conduit 3 is provided between the fluid reservoir 6 and the body interface 2. In addition to this, only a single pump 4 is provided.

Beyond this, an energy supply unit 16 (for instance a rechargeable or substitutable battery) is provided to supply the components of the device of FIG. 1 with electrical energy.

As shown in FIG. 1, the described embodiment of the invention uses a single fluidic conduit 3 between the body 1 and the therapy device 17 for both the insulin 8 supply as also for glucose measurement.

The system 17 comprises the fluid reservoir 6 which may include the insulin 8. Furthermore, the single pump 4 is provided, as well as the fluidic channel 3, the glucose measurement unit 7, the calibrator 5 (for instance an electrical conductivity measurement unit), the energy supply 16, the controller 15, and the body interface 2 which is capable to extract body fluid as well as to supply a medication.

The pump 4 moves perfusate fluid from the reservoir 6 through the fluidic channel 3 and via the body interface 2 into the body 1. When a fluid comprises also insulin 8, a variable amount of insulin 8 may be fed to the body 1 by varying the flow rate of the pump 4. When an information about the glucose level is desired, the pumping direction 13 of the pump 4 may be simply inverted. Then, the pump 4 pumps a mixture of the body fluid and the reservoir fluid back into the fluidic channel 3. Here, by means of the conductivity (or more generally an endogenous or exogenous marker) measurement unit 5, the mixture degree between body fluid and reservoir fluid is measured. Using the glucose measurement unit 7, the glucose level in the extracted mixture is measured. In the controller 15, on the basis of this information, the (blood) glucose level may be calculated.

When the mixture degree, when extracting the fluid, is too low for an accurate determination of the glucose, the mixture in the fluidic channel 3 can be inserted again in the body 1 and can be brought to a better mixture with the body fluid. The mixture can then be retracted again from the body 1 and may be brought into the fluidic channel 3 for detecting the mixture degree and the glucose concentration, and so on.

The sensors 5 and/or 7 can work continuously or discontinuously. For example, it is possible to use multiple measurement strip arrays for the measurement of the glucose and the conductivity.

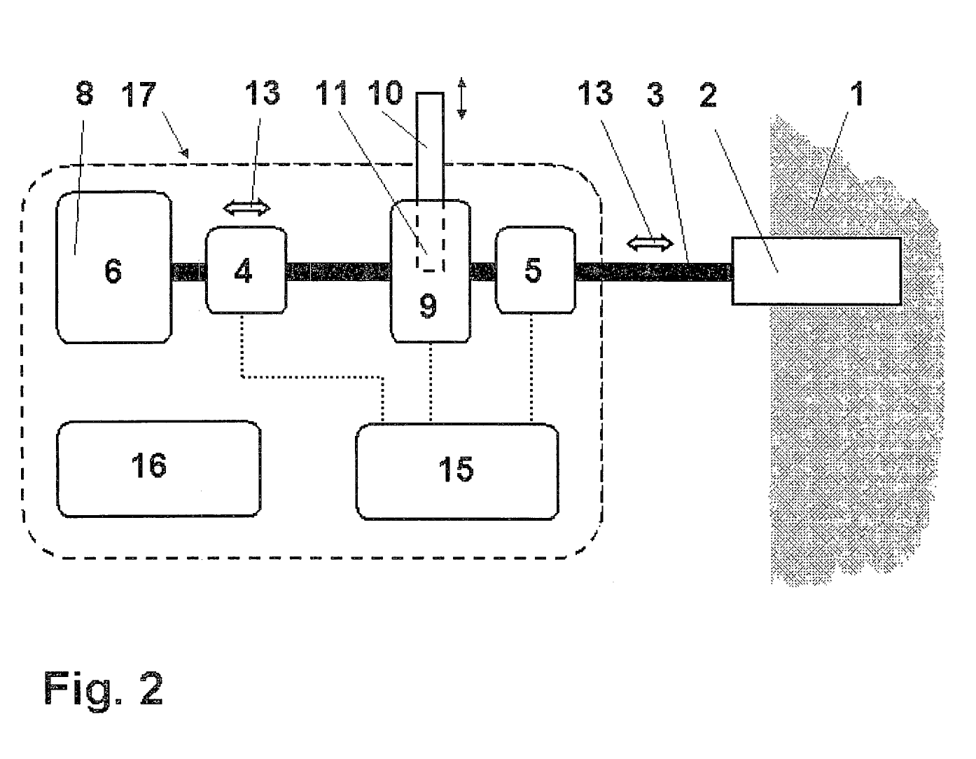

In the following, referring to FIG. 2, another device 17 according to an exemplary embodiment of the invention will be explained.

In the embodiment of FIG. 2, a discontinuous glucose measurement system is shown in which a test strip 10 is removably inserted from an exterior position (for instance by a human user) into the therapy device 17. In a coupling unit 9, a glucose reagent agent 11 is brought in functional connection with the extracted mixture, and the glucose level is measured. In the shown embodiment, the conductivity (or more generally an endogenous or exogenous marker) measurement 5 is performed (quasi-)continuously.

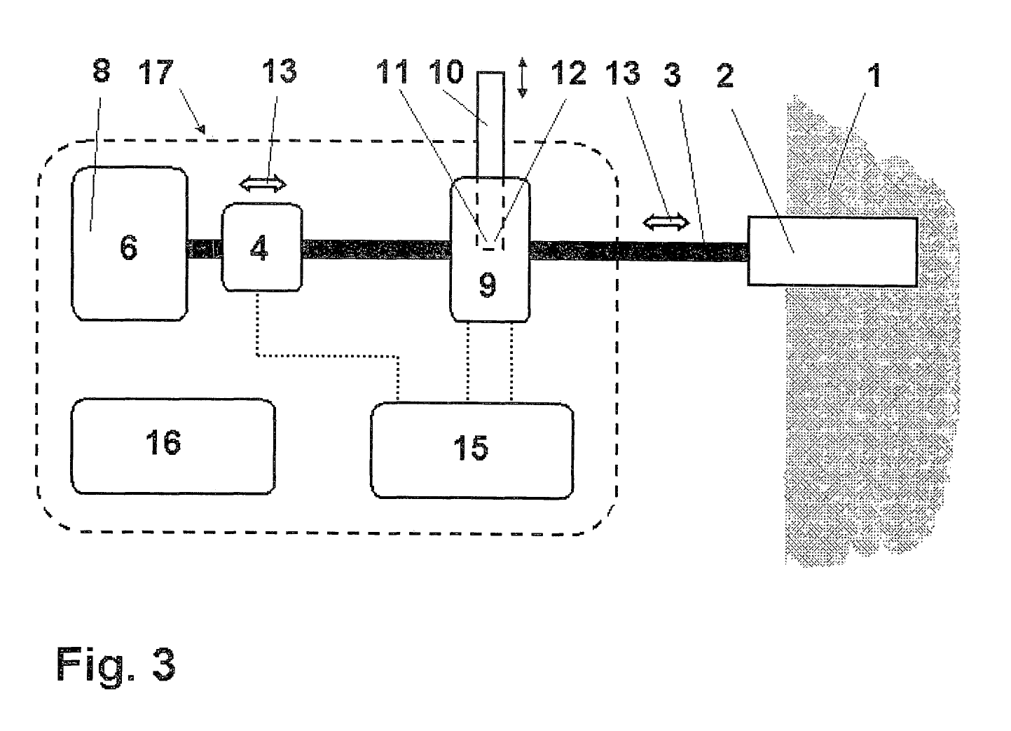

Referring to the embodiment shown in FIG. 3, as in the embodiment of FIG. 2, a measurement strip 10 is inserted from an exterior position into the therapy device 17 and is brought in connection with the extracted mixture in the couple unit 9. In contrast to the embodiment of FIG. 2, the measurement strip 10 comprises the glucose reagent agent 11 and a reagent agent 12 measuring the endogenous/exogenous marker, such as the conductivity. Thus, unit 5 may be omitted according to FIG. 3.

In the embodiment of FIG. 4, a continuous glucose and conductivity measurement unit is attached at the body interface 2, see units 5 and 7. In this scenario, the therapy device 17 can be constructed in a manner that insulin 8 is infused via the fluidic channel 3 (see reference numeral 14), or body fluid may be sucked (see reference numeral 13) back into the device 17. Thus, it is possible to simply inject the perfusate comprising the insulin 8 into the body 1 and to measure the glucose level without the need to suck back the liquid (see arrow 14). Since a (back-)flow of the perfusate between tissue and a surface of the body interface 2 is possible, the environment around the sensor 7 can be “diluted”. This dilution or equilibration or mixture degree can be measured by a marker, and the glucose measurement may be corrected accordingly. It is also possible to stop the infusion of the perfusate, it is possible to wait until equilibration, and then the glucose can be measured. Sucking back the perfusion fluid in the body interface 2 would then be provided for accelerating the equilibration procedure. (see arrow 13).

In the following, referring to FIG. 5A to FIG. 9C, exemplary embodiments for body interfaces 2 will be explained.

FIG. 5A and FIG. 5B show a transcutaneous catheter 2 having an insertion needle 21, a plastic cannula 22 and a septum 24.

FIG. 5A shows the system in a configuration in which the insertion needle 21 is still part of the device 2. In FIG. 5B, the insertion needle 21 has been removed.

Referring to the embodiment shown in FIG. 6A and FIG. 6B, in order to increase the efficiency of the extraction, additionally perforations 25 or holes may be provided in a wall of the plastic cannula 22.

Again, FIG. 6A shows the system in a state in which the insertion needle 21 is still within the device 2, whereas the insertion needle 21 has been removed in FIG. 6B.

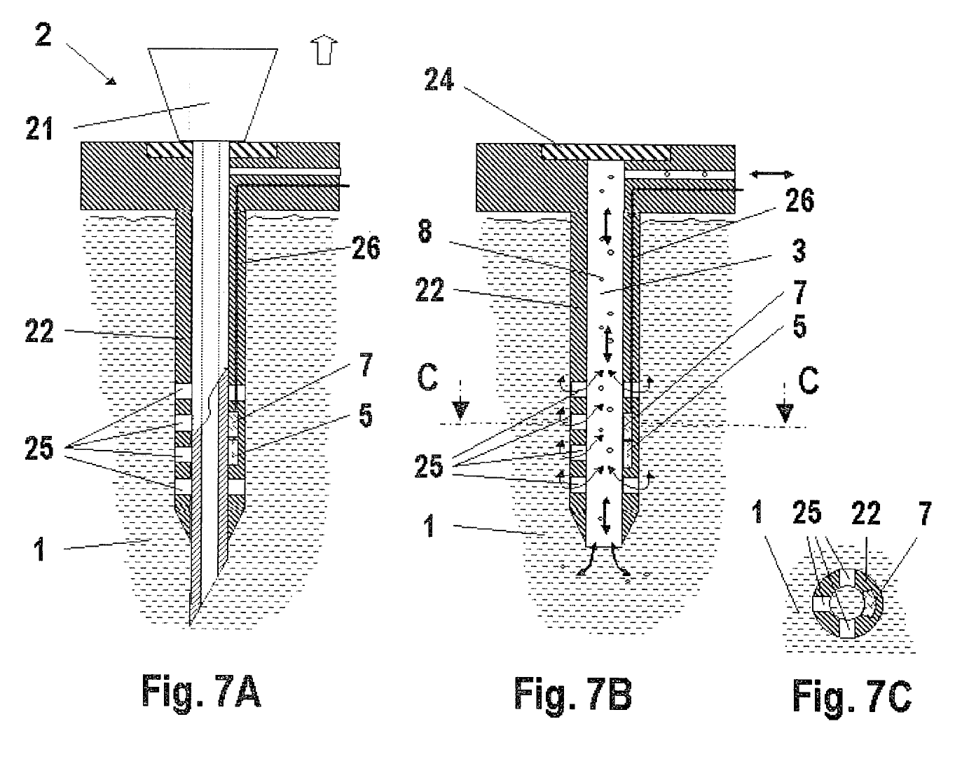

FIG. 7A to FIG. 7C show a body interface 2 in which the glucose sensor 7 and the conductivity (or more generally an endogenous or exogenous marker) sensor 5 are integrated at an interior wall of the plastic cannula 22 so as to be in contact with the fluid being located within the fluidic channel 3. A signal line 26 which is integrated in the wall of the plastic cannula 22 as well transmits the sensor signals to the therapy device 17.

FIG. 7A shows the device 2 in a configuration in which the insertion needle 21 is still within the device 2, in FIG. 7B the insertion needle 21 has been removed, and FIG. 7C shows a cross-section along a line C-C according to FIG. 7B.

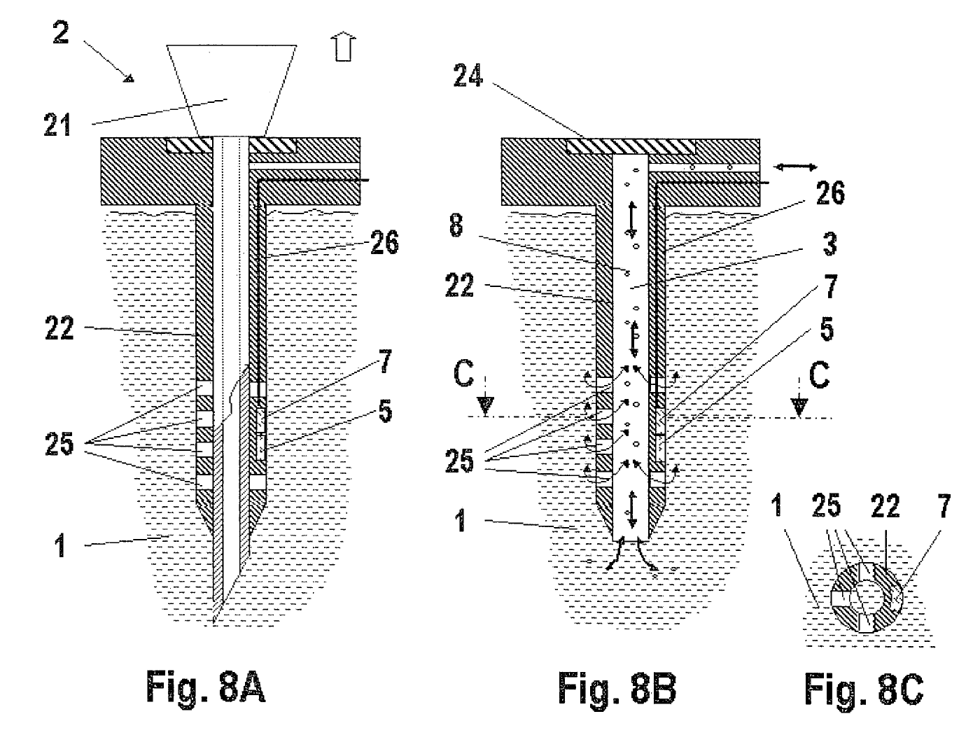

FIG. 8A to FIG. 8C shows a body interface 2 in which the glucose sensor 7 and the conductivity (or more generally an endogenous or exogenous marker) sensor 5 are integrated at an exterior wall of the plastic cannula 22 so as to be in contact with the fluid which is between the tissue 1 and the wall of the plastic cannula 22. For a glucose measurement, the supply of insulin 8 may be interrupted and body fluid may be sucked using the pump 4. In this context, the fluidic mixture positioned between the tissue 1 and the wall of a plastic cannula 22 will be sucked away so that the glucose sensor 7 is in tight contact with a pure non-diluted body fluid.

FIG. 8A shows an operation state in which the insertion needle 21 is still part of the device 2, in FIG. 8B the insertion needle 21 has been removed, and FIG. 8C shows a cross-section along a line C-C of FIG. 8B.

FIG. 9A to FIG. 9C are very similar to the embodiment of FIG. 8A to 8C and show the same procedure with a catheter in which no additional holes are provided in the wall of the plastic cannula 22. In such a configuration, it is possible to switch the pump 4 a plurality of times so as to perform a cycle or sequence of multiple pumping and suction procedures.

FIG. 10A and FIG. 10B show an embodiment of the invention in which a miniaturized insulin pump (patch pump) comprises a continuous conductivity measurement 5. From an exterior position, a measurement strip 10 is inserted via the septum 24 in such a manner that the glucose reagent agent 11 is in contact with the sucked fluid in the fluidic channel 3. The evaluation device 27 then transmits telemetrically the glucose value to the control unit 15. Such a data transmission may be performed in a wireless manner, for instance by Bluetooth, infrared radiation, an RFID reader/sender system, or the like. It is also possible that such a transmission occurs in a wired manner, for instance via a cable.

It should be noted that the term “comprising” does not exclude other elements or steps and the “a” or “an” does not exclude a plurality. Also elements described in association with different embodiments may be combined.

It should also be noted that reference signs in the claims shall not be construed as limiting the scope of the claims.

1. A device for determining a value of a physiological parameter of a body fluid of a body under investigation, the device comprising
a fluid reservoir for containing a perfusate fluid;
a body interface insertable into the body under investigation for a fluid communication between the fluid reservoir and the body fluid;
a fluid conduit between the fluid reservoir and the body interface;
a bidirectional fluid transport unit for transporting the perfusate fluid from the fluid reservoir through the fluid conduit and the body interface into the body under investigation and for subsequently transporting a mixture of the perfusate fluid and the body fluid from the body under investigation into the body interface; and
a sensor for sensing the value of the physiological parameter based on an analysis of the body fluid, particularly based on an analysis of the mixture of the perfusate fluid and the body fluid; wherein the device is adapted for supplying a physiologically active substance to the body under investigation; wherein the fluid reservoir contains the perfusate fluid including the physiologically active substance.

2. (canceled)

3. (canceled)

4. The device of claim 1, comprising a control unit adapted for controlling the bidirectional fluid transport unit for transporting the perfusate fluid from the fluid reservoir through the fluid conduit and the body interface into the body under investigation and for subsequently transporting the mixture of the perfusate fluid and the body fluid from the body under investigation into the body interface.

5. The device of claim 4, wherein the control unit is adapted for controlling the bidirectional fluid transport unit for transporting the mixture of the perfusate fluid and the body fluid from the body under investigation into the body interface only upon detecting that the perfusate fluid and the body fluid have been mixed sufficiently so as to exceed a predetermined mixture degree.

6. The device of claim 4, wherein the control unit is adapted for controlling the bidirectional fluid transport unit to switch a plurality of times between an operation state in which the perfusate fluid is transported in a direction from the body interface into the body under investigation, and an operation state in which the mixture of the perfusate fluid and the body fluid is transported in an opposite direction from the body under investigation into the body interface.

7. The device of claim 1, wherein the device comprises exactly one fluid conduit between the fluid reservoir and the body interface.

8. The device of claim 1, wherein the device comprises a bifurcation-free fluid conduit between the fluid reservoir and the body interface.

9. The device of claim 1, wherein the sensor is positioned within the fluid conduit.

10. The device of claim 1, wherein the sensor is positioned at a wall of the body interface, particularly at an interior wall or at an exterior wall of the body interface.

11. The device of claim 1, wherein the body interface comprises a wall having a plurality of fluid-exchange perforations.

12. The device of claim 1, comprising a single bidirectional fluid transport unit.

13. The device of claim 1, wherein a fluid transport direction of the bidirectional fluid transport unit is invertible.

14. The device of claim 1, comprising a calibration unit adapted for determining a mixture degree of the perfusate and the body fluid.

15. The device of claim 14, wherein the calibration unit is positioned at a wall of the body interface, particularly at an interior wall or at an exterior wall of the body interface.

16. The device of claim 4, wherein the control unit is adapted to determine an amount of the physiologically active substance to be delivered to the body under investigation based on the value of the physiological parameter sensed by the sensor.

17. The device of claim 14, wherein the control unit is adapted to determine an amount of the physiologically active substance based on the mixture degree determined by the calibration unit.

18. The device of claim 14, wherein the control unit is adapted to accept or reject to determine the amount of the physiologically active substance based on the mixture degree determined in the calibration unit, particularly by measuring an exogenous and/or an endogenous marker.

19. The device of claim 1, wherein the body interface is adapted to extract the mixture of the perfusate fluid and the body fluid from the body under investigation.

20-33. (canceled)

34. A method of determining a value of a physiological parameter of a body fluid of a body under investigation, the method comprising
inserting a body interface into the body under investigation for enabling a fluid communication between the body fluid and a fluid reservoir containing a perfusate fluid;
transporting, by a bidirectional fluid transport unit, the perfusate fluid from the fluid reservoir through a fluid conduit and the body interface into the body under investigation;
subsequently transporting a mixture of the perfusate fluid and the body fluid from the body under investigation into the body interface;
sensing the value of the physiological parameter based on an analysis of the body fluid, particularly based on an analysis of the mixture of the perfusate fluid and the body fluid; and
supplying a physiologically active substance to the body under investigation; wherein the fluid reservoir contains the perfusate fluid including the physiologically active substance.

35. A device for determining a value of a physiological parameter of a body fluid of a body under investigation, the device comprising
a fluid reservoir for containing a perfusate fluid;
a body interface insertable into the body under investigation for a fluid communication between the fluid reservoir and the body fluid;
a fluid conduit between the fluid reservoir and the body interface;
a fluid transport unit for transporting the perfusate fluid from the fluid reservoir through the fluid conduit and the body interface into the body under investigation;
a sensor positioned at the body interface and adapted for sensing the value of the physiological parameter based on an analysis of the body fluid, particularly based on an analysis of the mixture of the perfusate fluid and the body fluid;
a calibration unit positioned at the body interface and adapted for determining a mixture degree of the perfusate and the body fluid.

36. The device of claim 35, wherein the sensor is adapted to accept or reject to sense the value of the physiological parameter based on the mixture degree determined in the calibration unit.
